(19) United States
(12) Patent Application Publication
(10) Pub. No.: US 2025/0169982 A1
(43) Pub. Date: May 29, 2025

(54) HAIR LOSS PREVENTING DEVICE FOR ANTICANCER TREATMENT

(71) Applicant: NATIONAL CANCER CENTER, Goyang-si (KR)

(72) Inventors: Keun Seok LEE, Goyang-si (KR); Sung Hoon SIM, Goyang-si (KR); Heejung CHAE, Goyang-si (KR)

(73) Assignee: NATIONAL CANCER CENTER, Goyang-si (KR)

(21) Appl. No.: 18/839,816

(22) PCT Filed: Jan. 13, 2023
(86) PCT No.: PCT/KR2023/000627

§ 371 Date: Aug. 20, 2024

(30) Foreign Application Priority Data
Feb. 23, 2022 (KR) 10-2022-0023413
Jan. 10, 2023 (KR) 10-2023-0003194

(51) Int. Cl. A61F 7/00

(52) U.S. Cl. CPC A61F 7/007; A61F 2007/0008; A61F 2007/0075; A61F 2007/0078; A61F 2007/0091; A61F 2007/0094; A61F 2007/0096

(57) ABSTRACT

A hair loss prevention device for anticancer treatment that utilizes cooling and air pressure to prevent hair loss by inhibiting blood flow to the head. The device includes an air pressure pad, a heat dissipation pad, a thermoelectric element pad, and a cooling heat conduction pad positioned in that order on the inner side of a helmet housing that has a hemispherical shape with an empty interior and is configured to cause the heat dissipation pad, the thermoelectric element pad, and the cooling heat conduction pad to move toward the head by expansion of the air pressure pad, thereby compressing the head as a whole, and to transfer coldness generated by the thermoelectric element to the head through the cooling heat conduction pad. The device has a hair loss prevention effect by slowing down the blood flow rate.

### TECHNICAL FIELD

The following description relates to a device for preventing hair loss in cancer patients undergoing chemotherapy, and in particular, to a device for preventing hair loss in cancer patients undergoing chemotherapy which can be expected to prevent hair loss by doubly reducing the degree of blood circulation in the head by using cooling heat and air pressure, and which can be easily worn while being carried.

### BACKGROUND ART

Regardless of age or gender, there is a great deal of concern and interest in hair loss, and research into hair loss prevention has been ongoing for a long time. A wide variety of methods are being studied to prevent hair loss, including chemical treatments such as drugs, using lasers, massage devices using ultrasonic vibrations, red light, and millimeter waves. Recently, there are also methods using functional shampoos, etc.

An example of a conventional method for preventing hair loss is U.S. Pat. No. 6,156,059 (prior patent). The prior patent discloses the configuration of a head cooler that prevents hair loss by cooling the head. However, the prior patent must be provided with a flow passage through which a cooling fluid flows on a cover forming an inner heat exchange surface applied to the head to cool the head. Therefore, it is essential to configure a connecting tube for supplying the cooling fluid and the fluid. The bulkiness of the head cooler makes it difficult to use and store.

In particular, if the user using the head cooler is a patient receiving anticancer chemotherapy (cancer patient), using it can be inconvenient and cumbersome. Most patients receiving anticancer treatment experience hair loss, and because it is equipped with connecting tubes to supply and discharge cooling fluid, it was not easy to move around while wearing the head cooler.

Conventional devices for preventing hair loss, including those in prior patents, were structured to focus on only one function and had limited anti-hair loss performance.

### DISCLOSURE

### Technical Problem

An aspect of the present invention is to provide a hair loss prevention device for anticancer treatment that can be easily worn by patients receiving chemotherapy to prevent hair loss when undergoing anticancer chemotherapy.

Another aspect of the present invention is to provide a hair loss prevention device for anticancer treatment that can be easily carried and worn by cancer patients (patients on anticancer treatment) and can move freely even when worn.

Another aspect of the present invention is to provide a hair loss prevention device for anticancer treatment that enables efficient hair loss prevention by reducing blood flow to the head using air pressure and a thermoelectric cooling method.

Another aspect of the present invention is to provide a hair loss prevention device for anticancer treatment so that it can be easily stored and managed even in a small place when not in use.

The technical problems of the present invention are not limited to the technical problems mentioned above, and other technical problems not mentioned may be clearly understood by those skilled in the art from the following description.

### Solution to Problem

In order to achieve this purpose, in one general aspect, a hair loss prevention device for anticancer treatment includes a helmet housing in a hemisphere shape with an empty interior to be worn on head of a cancer patient (patient on anticancer treatment); an air pressure pad installed on an inner surface of the helmet housing; a heat dissipation pad installed on an inner surface of the air pressure pad; a thermoelectric element pad installed on an inner surface of the heat dissipation pad and having a plurality of thermoelectric elements; and a cooling heat conduction pad installed on an inner surface of the thermoelectric element pad. The heat dissipation pad, the thermoelectric element pad, and the cooling heat conduction pad are configured to move toward the head by expansion of the air pressure pad to pressurize the head and transfer coldness generated by the thermoelectric element to the head through the cooling heat conduction pad.

A check valve may be connected to the air pressure pad so that external air is injected at a constant pressure.

Inside the heat dissipation pad, a heat dissipation pipe for absorbing and exchanging heat generated by the thermoelectric element and may be installed. a diameter of an inlet and a diameter of an outlet of the heat dissipation pipe may be formed to be same or different.

A fan may be installed at the inlet of the heat dissipation pipe.

Insulating members may be provided between the thermoelectric elements.

The hair loss prevention device for anticancer treatment may further include a temperature sensor configured to detect temperature of the cooling heat conduction pad.

The helmet housing may include a controller and a battery mounting part inside. The helmet housing may be provided with a power terminal for external power use; an operation part equipped with a power button, a temperature control button, an air pressure control button, and a timer setting button for use of a hair loss prevention device for anticancer treatment; and a lighting part for displaying colors according to a temperature of the cooling heat conduction pad exposed to outside.

The hair loss prevention device for anticancer treatment may further include a remote controller connected to the controller by wire or wirelessly.

The controller may be configured to control a temperature of the cooling heat conduction pad to be between 15° C. and 20° C. as a result of the detection of the temperature sensor.

The controller may increase a coldness generation capacity of the thermoelectric element when the temperature of the cooling heat conduction pad exceeds 20° C. and decrease the coldness generating ability of the thermoelectric element when the temperature of the cooling heat conduction pad is less than 15° C.

The hair loss prevention device for anticancer treatment may reduce blood circulation in the head by pressurizing the head by air pressure and by coldness transported to the head before, during, and after anticancer treatment.

### Effects of Invention

According to the present invention, the function of pressurizing the head and the function of transporting appropriate coldness to the head are applied together to further reduce head blood flow, thereby improving hair loss prevention performance compared to existing hair loss prevention devices.

According to the present invention, it is a helmet-shaped device worn on the head, and there is no separate equipment or device connected to the helmet, so cancer patients undergoing chemotherapy can move even while wearing a hair loss prevention device. It can be used freely and conveniently, and its anti-hair loss effect improves the quality of life of patients receiving anticancer treatment. Its small size makes it easy to carry, and it is easy to store and manage when not in use.

### BRIEF DESCRIPTION OF DRAWINGS

FIG. 1 is a view illustrating an example of wearing a hair loss prevention device for anticancer treatment according to an embodiment of the present invention.

FIG. 2 is a cross-sectional view of the hair loss prevention device for anticancer treatment in FIG. 1.

FIG. 3 is a block diagram of the hair loss prevention device for anticancer treatment in FIG. 1.

FIG. 4 is a flow diagram illustrating the use of the hair loss prevention device for anticancer treatment according to the present invention.

### BEST MODE FOR IMPLEMENTATION OF THE INVENTION

Hereinafter, preferred embodiments of the present invention will be described in detail with reference to the accompanying drawings. Advantages and features of the present invention and a method of achieving them will become apparent with reference to the embodiments described below in detail with the accompanying drawings. However, the present invention is not limited to the embodiments disclosed below, but will be implemented in various forms, and only the present embodiments are provided to ensure that the disclosure of the present invention is complete, to fully inform those of ordinary skill in the art to which the present invention belongs, and the present invention is only defined by the scope of the claims. Hereinafter, the same reference numerals refer to the same elements.

Although first, second, etc. are used to describe various devices, components, and/or sections, these devices, components, and/or sections are not limited by these terms. These terms are used only to distinguish one device, component, or section from other devices, components, or sections. Therefore, it goes without saying that the first element, the first element, the first element, or the first section mentioned below may be a second element, a second element, or a second section within the technical idea of the present invention.

The terms used in the present invention are used only to describe a specific embodiment and are not intended to limit the present invention. Singular expressions include plural expressions unless the context clearly indicates otherwise. In the present application, the term “comprise” or “made of” is intended to specify that there is a feature, number, step, operation, component, part, or combination thereof described in the specification, and it should be understood not to preclude the presence or additional possibility of one or more other features or numbers, steps, operations, components, parts, or combinations thereof.

Spatially relative terms such as below, beneath, lower, above, upper, etc. facilitate the correlation between one element or component and other elements or components as shown in the drawing. It can be used to describe. Spatially relative terms should be understood as terms that include different directions of the element during use or operation in addition to the direction shown in the drawings. For example, when an element shown in a drawing is turned over, an element described as below (below, beneath) another element may be placed above (upper) the other element. Accordingly, the illustrative term below may include both downward and upward directions. Elements can also be oriented in other directions, so spatially relative terms can be interpreted according to orientation.

As used in the present invention, expressions indicating a part such as “unit” or “part” mean that the corresponding component is a device that can include a specific function, software that can include a specific function, or a device that can include a specific function. It means that it can represent a combination of and software, but it cannot be said that it is necessarily limited to the expressed functions. This is only provided to help a more general understanding of the present invention, and is provided to those with ordinary knowledge in the field to which the present invention pertains. Various modifications and variations are possible from this description.

In addition, it should be noted that all electrical signals used in the present invention are examples, and if an inverter or the like is additionally provided in the circuit of the present invention, the signs of all electrical signals to be described below may be reversed. Therefore, the scope of the present invention is not limited to the direction of the signal.

Accordingly, the spirit of the present invention should not be limited to the described embodiments, and the scope of the patent claims described below as well as all things that are equivalent or equivalent to the scope of this patent claim shall fall within the scope of the spirit of the present invention.

In the following, the invention will be described in more detail with reference to the accompanying drawings.

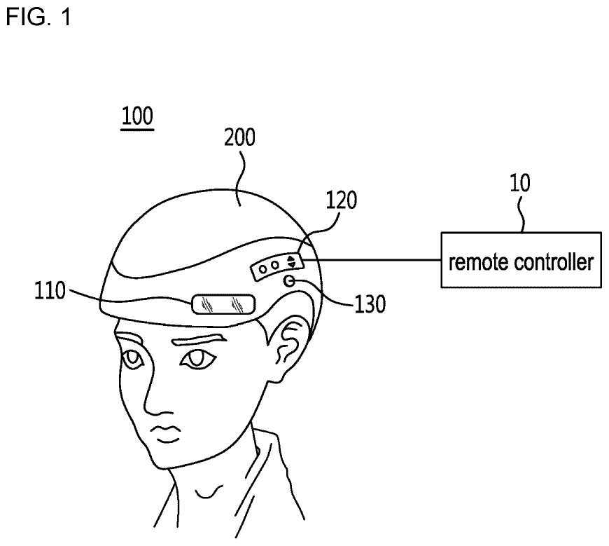

FIG. 1 is a view illustrating an example of wearing a hair loss prevention device for anticancer treatment according to an embodiment of the present invention.

As shown in the figures, a hair loss prevention device for anticancer treatment is a helmet structure that can be worn on the head of a user, i.e., a cancer patient being treated with chemotherapy. As it is a structure that can be easily worn on the head, it can be easily used not only by patients undergoing chemotherapy but also by the general public. In addition, as a helmet structure, the overall size is not large, making it easy to store and manage when not in use. In FIG. 1, the helmet structure covers only the head area, but the helmet structure may also cover the entire head, including the ears and the back of the neck, except for the face.

On the outer surface of a helmet housing 200 of the hair loss prevention device for anticancer treatment, a lighting part 110, an operation part 120, and a power terminal 130, which will be described below, are formed. A remote controller 10 in the form of a remote control connected to the helmet housing 200 by wire or wirelessly may be provided. The remote controller 10 is intended to allow the user to easily make and check adjustments while wearing the helmet. The remote controller 10 is configured by applying all the various components provided in the operation part 120.

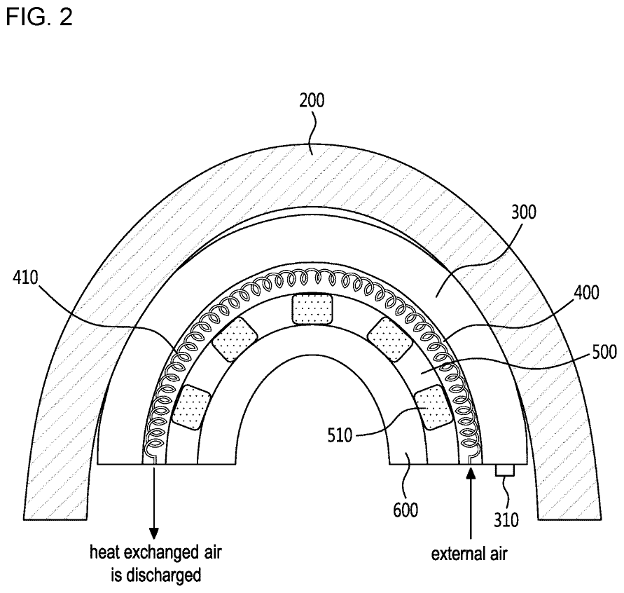

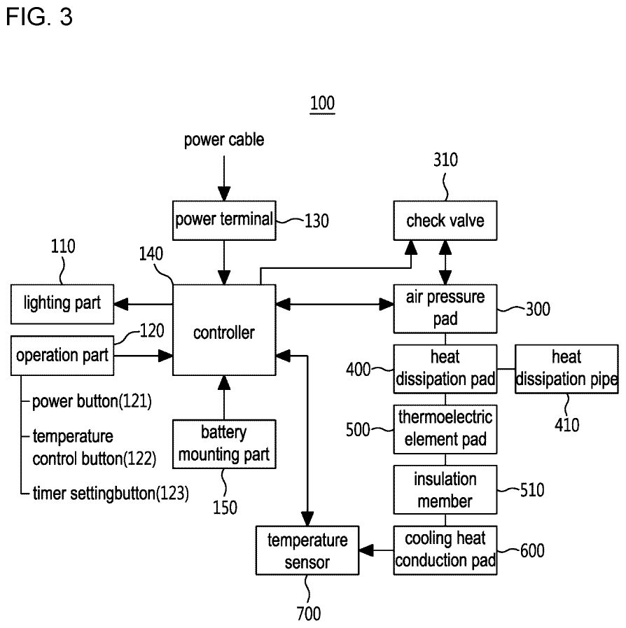

FIG. 2 is a cross-sectional view of the hair loss prevention device for anticancer treatment in FIG. 1. FIG. 3 is a block diagram of the hair loss prevention device for anticancer treatment in FIG. 1.

Referring to FIGS. 2 and 3 together, the hair loss prevention device for anticancer treatment 100 comprises a helmet housing 200 that forms an overall shape and is protected against external impacts. The helmet housing 200 may be formed in a roughly hemispherical shape with a hollow interior for wearing on the head of a patient on anticancer treatment.

The interior of the helmet housing 200 may be provided with a controller 140 that controls the operation of the hair loss prevention device for anticancer treatment 100 and a battery mounting part 150 where a rechargeable battery can be installed. The controller 140 may be a series of microprocessors or CPUs, and may be mounted on a PCB substrate together with various other circuit elements. In other words, a PCB substrate on which the controller 140 is mounted is installed inside the helmet housing 200. The power terminal 130 is formed so that commercial power can be used in addition to battery power. It is used by connecting a power cable to the power terminal 130.

On the outside of the helmet housing 200, an operation part 120 and a lighting part 110 are formed, as shown in FIG. 1. The operation part 120 is a part provided with various buttons or switches for use of the hair loss prevention device for anticancer treatment 100 and may be provided with a touch screen that can be operated by a touch method, if necessary. The operation part 120 includes a power button 121 for turning on/off, a temperature control button 122 for controlling the temperature of a cooling heat conduction pad 600, and a timer setting button for setting the operating time of the hair loss prevention device 100. Of course, there could be more buttons for other functions for ease of use.

The lighting part 110 is a part that displays colors according to the temperature of the cooling heat conduction pad 600. The temperature of the cooling heat conduction pad 600 can be easily checked just by the color displayed on the lightning part 110, and it can also be used as a mood lamp in a dark place. In addition, the lighting part 110 can also be used as a lantern to illuminate the front by employing LED elements. The lantern function can be useful when lighting a dark place or reading while wearing the hair loss prevention device 100.

An air pressure pad 300 that expands by injection of external air is installed inside the helmet housing 200. When air is injected in the normal (i.e., not in use) state before expansion, the volume increases and pressurizes the user's head. Additionally, a check valve 310 is provided on one side of the air pressure pad 300 so that external air can be injected at a constant pressure. As another example, the check valve 310 may be provided on the outer surface of the helmet housing 200. In other words, although not shown in the drawing, an air transport passage that penetrates the helmet housing 200 and is connected to the air pressure pad 300 is formed, and the check valve 310 is installed at one end of the air transport passage corresponding to the outer surface of the helmet housing 200. When the check valve 310 is unlocked while the air pressure pad 300 is expanded, the air inside the air pressure pad 300 is discharged to the outside.

As shown in FIG. 2, there is a heat dissipation pad 400 on the inner surface of the air pressure pad 300, a thermoelectric element pad 500 on the inner surface of the heat dissipation pad 400, and the cooling heat conduction pad 600 on the inner surface of the thermoelectric element pad 500 are composed in order. When the air pressure pad 300 expands, these pads 400, 500, and 600 also move toward the head. This provides the effect of pressurizing the head.

The heat dissipation pad 400 on the outside of the thermoelectric element pad 500 is for discharging heat generated by driving the thermoelectric element provided inside the thermoelectric element pad 500 to the outside. Accordingly, a heat dissipation pipe 410 for heat exchange with a thermoelectric element is installed inside the heat dissipation pad 400. The heat dissipation pipe 410 may be, for example, a tube of a predetermined diameter with an empty interior, and may be wound in a spiral coil shape. An inlet and an outlet are formed in the heat dissipation pipe 410, and external air introduced through the inlet passes along the heat dissipation pipe 410 to absorb heat generated in the thermoelectric element, exchange heat, and discharge the heat to the outside through the outlet. Therefore, the heat generated from thermoelectric elements can be efficiently discharged to the outside.

In this embodiment, a fan that rotates by a driving force such as a motor may be provided at the front end of the heat dissipation pipe 410 to allow external air to easily flow in. Alternatively, the inlet and outlet diameters of the heat dissipation pipe 410 may be designed to be different so that air can be introduced by pressure difference without driving force. Preferably, the inlet diameter may be larger than the outlet diameter.

The thermoelectric element pad 500 includes a plurality of thermoelectric elements, and in this embodiment, the thermoelectric elements may be Peltier thermoelectric elements using the Peltier effect. One side of the thermoelectric element generates coldness and the other side generates heat. The generated heat is discharged to the outside by the heat dissipation pad 400 mentioned above.

In this embodiment, an insulation member 510 is provided between each thermoelectric element of the thermoelectric element pad 500. As shown in the drawing, the insulation member 510 is provided between the heat dissipation pad 400 and the cooling heat conduction pad 600 to block heat transfer, and further serves to block heat transfer between thermoelectric elements.

The cooling heat conduction pad 600 serves to transfer coldness generated by the thermoelectric element to the head. Since this cooling heat conduction pad 600 is in direct with the user's head, it should be made of a material that is harmless to the skin.

A temperature sensor 700 for sensing the temperature of the cooling heat conduction pad 600 is included. Because maintaining the temperature of the cooling heat conduction pad 600 within a suitable range, for example, between 15° C. and 20° C., helps suppress blood circulation in the head, the controller 140 is configured to control the temperature of the cooling heat conduction pad 600 to maintain it within the predetermined temperature range. In other words, based on the sensing result of the temperature sensor 700, the controller 140 controls the operation of the thermoelectric elements to ensure that the temperature of the cooling heat conduction pad 600 is always maintained at the optimal temperature. Therefore, if the temperature of the cooling heat conduction pad 600 exceeds 20° C., the thermoelectric element's cooling capacity is increased, whereas if the temperature of the cooling heat conduction pad 600 falls below 15° C., the cooling capacity of the thermoelectric element is reduced.

In FIG. 2, the thicknesses of the air pressure pad 300, the heat dissipation pad 400, the thermoelectric element pad 500, and the cooling heat conduction pad 600 may all be designed to be the same or different. For example, depending on the treatment period of a cancer patient, the pressure applied to the head by the air pressure pad 300 can be increased or the coldness transfer can be designed to be the greatest through the cooling heat conduction pad 600. In this case, the thickness of the pads 300, 400, 500, and 600 may each be designed and configured differently.

### Mode for Implementation of the Invention

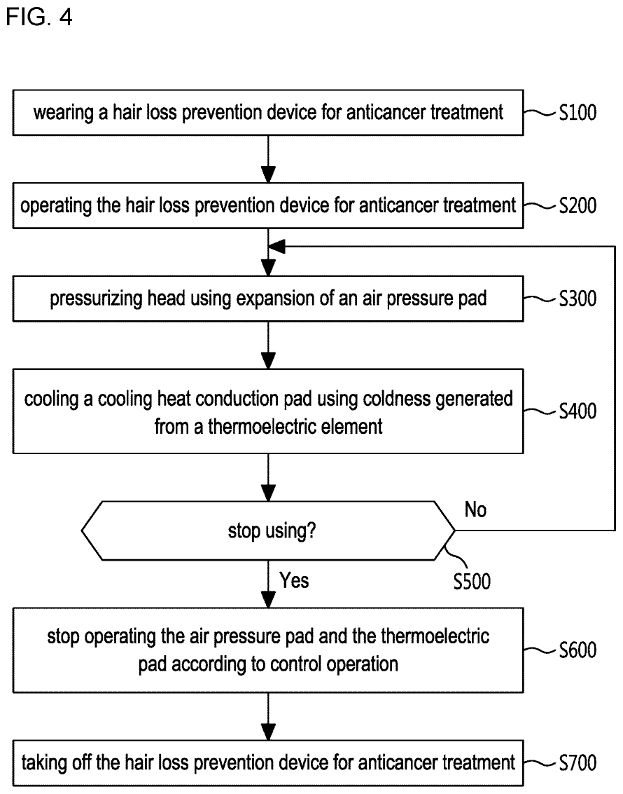

In the following, an example of the use of the hair loss prevention device for anticancer treatment as described above will be described with reference to FIG. 4.

FIG. 4 is a flowchart explaining the use state of the hair loss prevention device for anticancer treatment according to the present invention.

The user wears the hair loss prevention device for anticancer treatment 100 on the head (S100).

In such a state, the power button 121 and the temperature control button 122 of the operation part 120 are operated to maintain the desired temperature of the cooling heat conduction pad 600 while driving the hair loss prevention device for anticancer treatment 100 (S200). The operation of the hair loss prevention device for anticancer treatment 100 is performed by a user by operating the operation part 120 or the remote controller 10.

The hair loss prevention device for anticancer treatment 100 according to the present invention may be worn and used by cancer patients before, during, or after receiving anticancer chemotherapy, or for a predetermined amount of time from before to after receiving anti-cancer chemotherapy.

When the hair loss prevention device for anticancer treatment 100 starts to operate, as in step S300, the controller 140 allows air to be injected from the outside at an appropriate pressure through the check valve 310 of the air pressure pad 300. The injection of external air is performed for a certain period of time, and is stopped when the air pressure pad 300 reaches a preset inflation state. When the air pressure pad 300 expands through this process, the heat dissipation pad 400, the thermoelectric element pad 500, and the cooling heat conduction pad 600 provided inside the air pressure pad 300 during the expansion operation all move in the direction of the head. Therefore, the cooling heat conduction pad 600, which is in direct contact with the head, presses the head at an appropriate pressure (S300).

While the cooling heat conduction pad 600 in step S300 is pressing the head, an operation of cooling the cooling heat conduction pad 600 provided on the inside is performed simultaneously due to the operation of the thermoelectric element (S400). Cooling heat is directly transferred to the head by cooling of the cooling heat conduction pad 600. At this time, since the heat generated as the thermoelectric element is driven is discharged to the outside through heat exchange with external air flowing through the heat dissipation pipe 410, the temperature inside the helmet housing 200 will not rise and maintain an appropriate temperature. When the cooling heat conduction pad 600 is cooled, the temperature sensor 700 senses the temperature of the cooling heat conduction pad 600. According to the sensing result, the controller 140 increases or decreases the coldness generation capacity of the thermoelectric element so that the cooling heat conduction pad 600 continuously maintains an appropriate temperature while the hair loss prevention device 100 is driven.

In this way, since the expansion of the air pressure pad 300 and the cooling of the cooling heat conduction pad 600 are applied together, the user's head is simultaneously affected by the pressurized state and the cold state by the coldness. Therefore, blood flow to the head may be reduced more than usual. This has the advantage of preventing hair loss in addition to the reduction of blood flow to the head with only one conventional function.

When the user wishes to stop using the hair loss prevention device for anti-cancer treatment 100 according to embodiments of the present invention (S500), the user may operate the operation part 120 or the remote controller 10. Therefore, when the power supply is cut off, the operation of the air pressure pad 300 and the thermoelectric element pad 500 will stop (S600) according to the control operation of the controller 140, and at this time, the check valve 310 of the air pressure pad 300 operates again to discharge the internal air to the outside. Due to air discharge, the internal pressure of the air pressure pad 300 is reduced, and the pressure pressing the head is also released. This allows the user to easily take off the hair loss prevention device for anti-cancer treatment 100. In other words, if the air pressure pad 300 continues to remain inflated, it is not easy to take off the hair loss prevention device for anticancer treatment 100 because it is pressuring the head.

The use of the hair loss prevention device for anticancer treatment 100 may be stopped by setting the time set by the timer setting button 123. The timer is set before use, and when the set time arrives during use, the controller 140 stops driving the air pressure pad 300 and the thermoelectric element pad 500, allowing the hair loss prevention device for anticancer treatment to be taken off.

In this way, the invention prevents hair loss by using the two functions of air pressure and cooling heat together as a way to reduce blood flow to the head, and as a helmet structure worn on the head, not only the general public but also patients receiving chemotherapy can move freely and use it.

While the above has been described with reference to the illustrated embodiments of the invention, they are exemplary only, and it will be apparent to one having ordinary skill in the art to which the invention belongs that various modifications, changes, and equivalents are possible without departing from the spirit and scope of the invention. The true scope of technical protection of the invention should therefore be determined by the technical ideas of the appended claims.

### INDUSTRIAL APPLICABILITY

It can be used as a hair loss prevention device for patients undergoing anticancer treatments.

1. A hair loss prevention device for anticancer treatment, comprising:
a helmet housing having a hemisphere shape with an empty interior configured to be worn on a head of a patient on anticancer treatment;
an air pressure pad installed on an inner surface of the helmet housing;
a heat dissipation pad installed on an inner surface of the air pressure pad;
a thermoelectric element pad installed on an inner surface of the heat dissipation pad and having a plurality of thermoelectric elements; and
a cooling heat conduction pad installed on an inner surface of the thermoelectric element pad,
wherein the heat dissipation pad, the thermoelectric element pad, and the cooling heat conduction pad are configured to move toward the head by expansion of the air pressure pad to pressurize the head and transfer coldness generated by the thermoelectric element to the head through the cooling heat conduction pad.

2. The hair loss prevention device of claim 1,
wherein a check valve is connected to the air pressure pad so that external air is injected at a constant pressure.

3. The hair loss prevention device of claim 1,
wherein a heat dissipation pipe configured to absorb heat generated from the thermoelectric element and heat-exchange the heat is installed on an inner surface of the heat dissipation pad, and
wherein a diameter of an inlet and a diameter of an outlet of the heat dissipation pipe are formed to be the same or different.

4. The hair loss prevention device of claim 3,
wherein a fan is installed at the inlet of the heat dissipation pipe.

5. The hair loss prevention device of claim 1,
wherein insulating members are provided between the thermoelectric elements.

6. The hair loss prevention device of claim 1, further comprising:
a temperature sensor configured to detect a temperature of the cooling heat conduction pad.

7. The hair loss prevention device of claim 6,
wherein the helmet housing further comprises a controller and a battery mounting part mounted on an inner surface,
wherein the helmet housing further comprises a power terminal for external power use, an operation part equipped with a power button, a temperature control button, and a timer setting button, and a lighting part exposed to an outside surface of the helmet housing and configured to display colors according to a temperature of the cooling heat conduction pad, and
wherein the lighting part provides a lantern function.

8. The hair loss prevention device of claim 7, further comprising:
a remote controller connected to the controller by wire or wirelessly.

9. The hair loss prevention device of claim 7,
wherein the controller is configured to control a temperature of the cooling heat conduction pad of 15° C. to 20° C.

10. The hair loss prevention device of claim 9,
wherein the controller increases a coldness generation capacity of the thermoelectric elements when the temperature of the cooling heat conduction pad exceeds 20° C. and decreases the coldness generation capacity of the thermoelectric element when the temperature of the cooling heat conduction pad is less than 15° C.

11. The hair loss prevention device of claim 1,
wherein the hair loss prevention device reduces blood circulation in the head by pressurizing the head by air pressure and by coldness transported to the head before, during, and after anticancer treatment.
